(19) United States
(12) Patent Application Publication
(10) Pub. No.: US 2004/0096914 A1
(43) Pub. Date: May 20, 2004

(54) Substrates with stable surface chemistry for biological membrane arrays and methods for fabricating thereof

(76) Inventors: Ye Fang, Painted Post, NY (US); Anthony G. Frutos, Painted Post, NY (US); Joydeep Lahiri, Painted Post, NY (US)

Correspondence Address: CORNING INCORPORATED, SP-TI-3-1, CORNING, NY, 14831

(21) Appl. No.: 10/300,954

(22) Filed: Nov. 20, 2002

(51) Int. Cl. G01N 33/53

(52) U.S. Cl. 435/7.9

(57) ABSTRACT

The present invention provides a method for preparing a physically stable array of biological membranes, including membrane proteins, on a surface, and the resultant article of manufacture. The method comprises providing a substrate; creating either a polar surface or reactive surface by coating the substrate with a material that either: (1) enhances the stability of lipid spots during withdrawing through a water/air interface and washing and drying protocols; or (2) gives rise to minimal non-specific binding of a labeled target to a background surface, and high specific binding to a probe receptor in said membrane array, or (3) both; depositing an array of biological-membrane microspots on the substrate. The method may further comprise applying a reagent that includes a soluable protein to stabilize the biological membranes on the surface. Also provided is an article having biological-membrane microspots that are associated in a stable fashion with a substrate surface embodying these properties.

### CROSS-REFERENCE TO RELATED APPLICATIONS

U.S. patent application Ser. No. 09/854,786, filed May 14, 2001, and U.S. patent application Ser. No. 09/974,415, filed Oct. 9, 2001.

### FIELD OF THE INVENTION

The present invention pertains to biological membrane arrays, as well as methods for their fabrication and use. In particular, the invention relates to strategies and surface chemistries for stabilizing arrays of membranes and membrane proteins on a substrate.

### BACKGROUND

The fabrication of biological membrane arrays, in particular membrane-protein arrays, is challenging because of difficulties associated with preserving the correctly folded conformation of proteins in an immobilized state, and the tendency for non-specific binding of targets to these immobilized proteins. As a large fraction of drug targets are membrane bound proteins (e.g., G-protein coupled receptors, ion-channels, etc.), there is an impetus to develop tools for high-throughput screening against membrane bound proteins. Membrane proteins maintain their folded conformations when associated with lipids; therefore, to create arrays of such proteins it is important to first develop surfaces that support the binding of membranes. Bilayer-lipid membranes adsorbed onto solid supports, referred to as supported bilayer-lipid membranes, can mimic the structural and functional roles of biological membranes. (See Sackmann, E. Science 1996, 271, 43-48; Bieri, C. et al., Nature Biotech., 1999, 17, 1105-1108; Groves, J. T. et al., Science 1997, 275, 651-653; Lang, H. et al., Langmuir 1994, 10, 197-210; Plant, A. L. et al., Langmuir 1999, 15, 5128-5135; and Raguse, B. et al., Langmuir 1998, 14, 648-659.) These hybrid surfaces were developed to overcome the fragility of black lipid membranes while preserving aspects of lateral fluidity observed in native biological membranes.

Surfaces for binding lipid membranes can be broadly classified into five categories: (1) hydrophobic surfaces (e.g., self-assembled monolayers presenting terminal methyl groups); (2) hydrophilic surfaces (e.g., bare glass or other inorganic surfaces); (3) hybrid surfaces presenting amphiphilic anchor molecules, which contain both hydrophobic and hydrophilic portions that bind bilayer lipid membranes; (4) surfaces presenting “polymer cushions,” and (5) surfaces that present functional moieties that specifically bind certain molecules in the biological membranes. Hydrophobic surfaces, which support the adsorption of lipid monolayers, are of limited utility, as they cannot be used to incorporate membrane-spanning proteins. (Plant, A. L., Langmuir 1999, 15, 5128-5135) Hydrophilic surfaces, which bind bilayer-lipid membranes, on the other hand, are also of limited utility, as they can be used only to incorporate membrane-spanning proteins with extra-membrane domains that are less thick than the layer of adsorbed water (˜10 Å, ˜1 nm). (Groves, J. T. et al., Science 1997, 275, 651-653; and Groves, J. T. et al., Langmuir 1998, 14 3347-3350.) Hybrid surfaces presenting amphiphilic anchor molecules, in contrast, offer the potential to incorporate a wide variety of membranes-spanning proteins. (Lang, H. et al., Langmuir 1994, 10, 197-210; and Raguse, B. et al., Langmuir 1998, 14, 648-659.) The amphiphilic anchor molecules can bind a lipid bilayer offset from the substrate surface by a distance determined by the length of the hydrophilic moiety of the anchor molecules. Surfaces that present “polymer-cushions” create a supported lipid bilayer offset from a hard substrate surface by a polymer matrix, and are deformable. Deformable surfaces, such as those presenting flexible amphiphilic tethers or polymer-cushions, can be used to immobilize a wide variety of membranes-spanning proteins. Surfaces that present functional moieties permit oriented immobilization of biological membranes, and provide the ability to control the orientation of receptors in the membranes; this, in turn, can produce potentially higher specificity in binding assays.

Methods to create arrays of membranes would enable high-throughput screening of multiple targets against multiple drug-candidates. Arrays of membranes may be obtained by using two general approaches. According to a first approach, patterned substrate surfaces having membrane-binding and non-binding regions are incubated with solutions containing membranes or membrane proteins. In an example of this first approach, one can fabricate grids of titanium oxide on a glass substrate, as titanium oxide resists the adsorption of lipids (Boxer, S. G. et al. Science 1997, 275, 651-653; and Boxer, S. G. et al. Langmuir 1998, 14, 3347-3350). This first approach, however, is not well suited to microarray applications that require the printing of different membrane compositions, because the process requires careful alignment between the printer and the lipid-binding regions. Micro-pipetting techniques have been used to spatially address each corralled lipid-binding region (Cremer, P. S. et al., J. Am. Chem. Soc. 1999, 121, 8130-8131). These methods, however, are cumbersome.

According to a second approach, solutions of membrane or membrane-proteins are printed onto unpatterned membrane-binding surfaces. To make membrane arrays by printing membranes on unpatterned surfaces, it is necessary to confine the printed lipid molecules, membranes or membrane-embedded proteins to the printed areas without lateral diffusion of the membrane molecules to the unprinted areas. This spatial confinement may be accomplished by two distinct means: (1) covalent or affinity-directed immobilization (e.g., streptavidin-biotin, Ni-histidine; and (2) non-covalent immobilization. Since lateral diffusion of molecules within a cell membrane is a fundamental property of natural or real biological membranes, covalent immobilization of the entire membrane is not desirable for the fabrication of biomimetic supported membranes. Fortuitously, strong intermolecular interactions between lipid molecules lead to a self-limiting expansion and enables non-covalent confinement of the printed membrane to the printed area. Boxer et al. have demonstrated that it is possible to pattern lipids on glass surfaces by microcontact printing using poly-dimethylsiloxane (PDMS) stamps “inked” with phosphatidylcholine (PC). They attributed the lateral confinement of the lipids to the stamped regions, to the self-limiting expansion of PC membranes to about 106% of the original printed areas (Hovis, J. et al, Langmuir 2000, 16, 894-897). The methods used by Boxer et al., however, have certain limitations. First, Boxer and co-workers carried out the stamping of lipids on surfaces immersed under water (Hovis 2000). Second, lipids adsorbed on the bare-glass substrates used by Boxer and coworkers spontaneously desorbed when drawn through an air-water interface (Cremer 1999). Cremer et al., propose in WO 01/20330 the use of spatially addressed lipid bilayer arrays that remain submerged underwater to preserve the planar support structure. Such systems may not be practical for robust, high-throughput, microarray-based assays, since such arrays would also need to be able to withstand processes in an ambient air environment.

### SUMMARY OF THE INVENTION

The present invention overcomes the problems and disadvantages associated with prior art arrays by providing an array comprising a plurality of biological membrane microspots associated with a surface of a substrate that can be produced, used and stored, not only in an aqueous environment, but also in an environment exposed to air under ambient or controlled humidities. The present invention pertains, in part, to a method for preparing and fabricating biological membrane arrays on a variety of surfaces. The method comprises: providing a support substrate; creating either a polar surface or reactive surface on the support (i.e., a covalent or reactive surface providing amine—or thiol—or other reactive moieties, and binding sites for membrane proteins or other effectors embedded in the lipid-membranes); providing a solution of biological molecules including biological membranes with membrane proteins; and depositing an array of biological-membrane microspots on the support, wherein the microspots are associated in a stable fashion with the surface of the support. Hence, the microspots remain in defined locations and retain their biological functions in either a liquid or air environment, or both. Deposition of the biological membranes may be accomplished by printing under either ambient or controlled humidity conditions. Both the polar surface and reactive surface can be created by coating the substrate with a material that either: (1) enhances the stability of lipid spots during withdrawl through a water/air interface and washing and drying protocols; or (2) gives rise to minimal non-specific binding of a labeled target to a background surface, and high specific binding to a probe receptor in said membrane array, or (3) both.

Applying a coating having functional groups that specifically binds to biomolecules in biological membranes, such as a wheat germ agglutinin layer between a surface of the support and a lipid bilayer, may create the reactive surface. The resultant biological-membrane array is resistant to degradation due to the hydrophobic effects of air. The method in certain situations may further comprise applying a reagent, which may include a protein, to stabilize the biological membranes (e.g., lipid and membrane protein-associated lipid) in each microspot. The method will enable the printing or other deposition of biological membranes in a neat and ordered fashion, without the detriments, such as membrane solution bleeding, associated with currently available techniques. The present invention also pertains to articles or substrates, and kits or assemblies for biological membrane arrays prepared according to the method as described herein.

According to the invention, the reagent used to stabilize arrays or micro-arrays of biological membranes on the support may include water-soluble proteins that will not interfere with the binding domains of target membrane proteins or other functional molecules in the membranes arrayed on surfaces. Proteins, for example, like bovine serum albumin (BSA) can bind non-specifically to substrates such as bare glass, mica, gold, self-assembled monolayers of silanes and alkylthiols, or polymer-grafted surfaces. The bound proteins on the substrate tend to pack together closely to form monolayers around the membrane microspots in the microarray, thereby stabilizing the overall membrane microarray. The reagent may also include either a hydrophilic polymer or a positively or negatively charged polymer, such as carboxymethyldextran.

Preferably, microspots of the biological membranes comprise a membrane bound protein. Most preferably, the membrane bound protein is a G-protein coupled receptor (GPCR), a G protein, an ion channel, a receptor serine/threonine kinase, a receptor guanylate cyclase or a receptor tyrosine kinase.

For certain embodiments in which the biological membrane microspot comprises a GPCR, the GPCR may be oriented depending on the application of the array, such that a desired domain, i.e. extracellular or intracellular, faces the solution. For example, orientation of the GPCR with its extracellular domain facing the solution is preferred for applications related to screening of ligands. The orientation with the intracellular domain facing the solution is preferred for applications involving functional assays. The desired orientation can be accomplished using substrate surface modification techniques discussed in detail below.

In another embodiment, the GPCRs contained within the microspots include members of a single or several related subfamilies of GPCRs. These arrays are referred to as “family-specific arrays.” Additionally, some GPCRs are highly expressed in certain tissue types including tumor tissue. This information is used to create arrays of GPCRs having similar tissue distribution (tissue-specific arrays) or similar physiological/pharmacological roles (function-specific arrays).

The substrate for use in the array of the present invention can comprise glass, silicon, metal or polymeric materials. The substrate can be configured as a chip, a slide or a microplate.

In certain embodiments, the surface of the substrate is coated. Preferably, the coating is a material that enhances the affinity of the biological membrane microspot for the substrate. In an embodiment, a coating material confers a water-contact angle ranging from about 5° to about 80°. A preferred coating material confers a contact angle ranging from about 15° to 60°. A most preferred coating material confers a contact angle ranging from about 25° to 45°.

The coating material can be a silane, thiol, or a polymer (biological or synthetic). Preferably, when the material is a thiol, the substrate comprises a gold-coated surface. Preferably, the thiol comprises hydrophobic and hydrophilic moieties. Most preferably, the thiol is a thioalkyl compound that presents amine moieties.

Preferably, when the coating material is a silane, the substrate comprises glass. Preferably, the silane presents terminal moieties including, for example, hydroxyl, carboxyl, phosphate, sulfonate, isocyanato, glycidoxy, thiol, or amino groups. A preferred silane coating material is a silane presenting amine functional groups. A most preferred silane coating material is γ-aminopropylsilane (GAPS).

When the coating material is a polymer, the coating may form a loosely packed polymer layer referred to as a “polymer cushion.” Preferably the polymer presents amine functional moieties such as poly(ethyleneimine), poly-L-lysine, and poly-D-lysine. Alternatively, a surface presenting amine-reactive functional moieites such as isothiocyanate, NHS ester, epoxide, and anhydrides, etc. can be further modified with a molecule presenting more than one amine group (e.g., 1,6-hexanediamine) to form an amine-presenting surface. Similarly, a surface presenting other reactive groups such as thiol- and carboxylate-reactive groups can be further modified with a molecule containing both the group reacting one of these chemical groups and at least one amine group to form an amine presenting surface.

In an alternative embodiment, the coating material is a derivatized monolayer (or several monolayers), multilayer or polylayer having covalently bonded linker moieties. Most preferably, the monolayer comprises a thioalkyl compound or a silane compound. Preferably, the silane- or thiol-derivatized surface can be further modified with one or more reagents (e.g. cationic polymers such as poly(diallydimethylammonium chloride, or glutaraldehyde) to enable membrane immobilization through either covalent or non-covalent bond formation.

Additional features and advantages of the present method and array device will be disclosed in the following detailed description. It is understood that both the foregoing summary and the following detailed description and examples are merely representative of the invention, and are intended to provide an overview for understanding the invention as claimed.

### BRIEF DESCRIPTION OF FIGS.

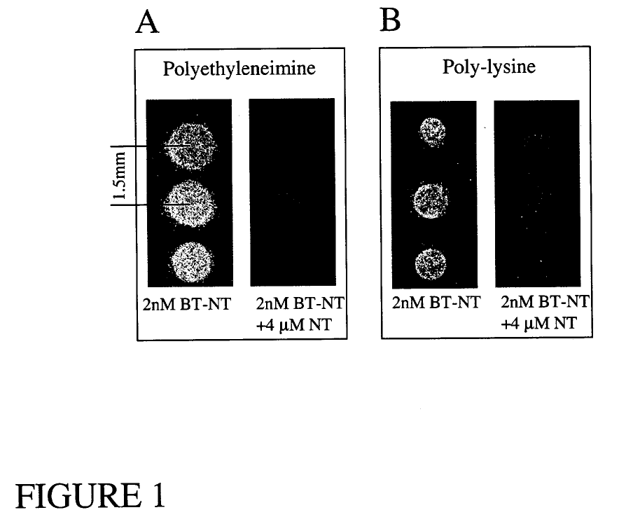

FIGS. 1A and B show fluorescence images of GPCR arrays, in which arrays of human neurotensin receptor subtype I (NTR1) were printed on hydrophilic polymer coated glass slides and incubated with the fluorescently labeled ligand neurotensin (2 nM Bodipy-TMR-NT, BT-NT) in the absence and presence of unlabeled neurotensin (4 μM). FIG. 1A shows NTR1 arrays on a polyethyleneimine-coated glass slide, and FIG. 1B shows NTR1 arrays on a poly-lysine-coated glass slide.

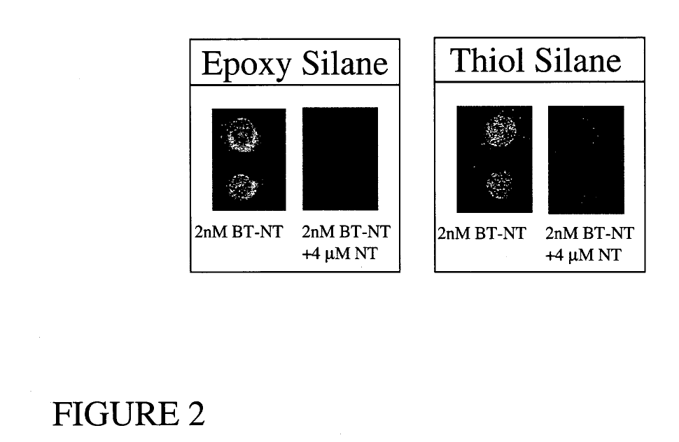

FIGS. 2A and B depict fluorescence images of GPCR arrays prepared like those of FIGS. 1A and B. FIG. 2A shows NTR1 arrays on an expoxy-silane-coated glass slide, while FIG. 2B shows NTR1 arrays on a thiol-silane-coated glass slide.

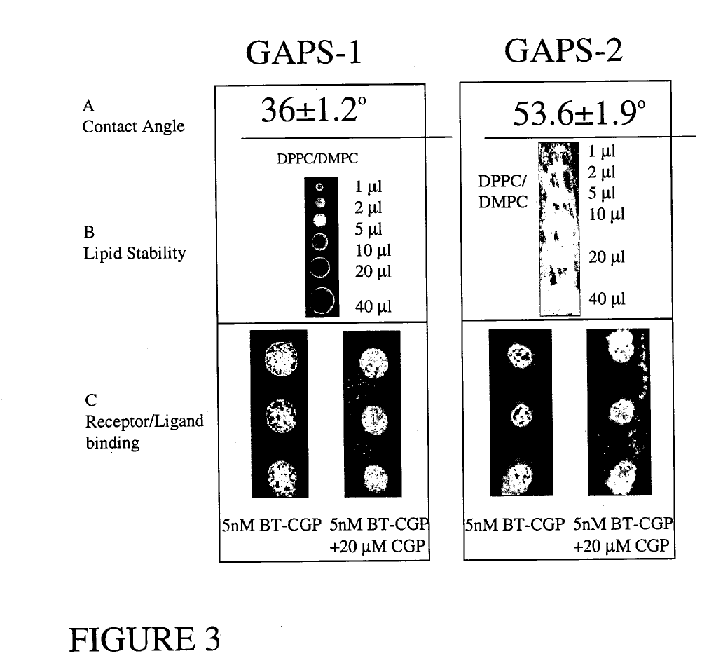

FIG. 3 shows a comparison of two different GAPS-coated slides. FIG. 3A presents the respective water-contact angles. FIG. 3B shows respective fluorescence images of DPPC/DMPC (4:1 in mole ratio) doped with 4% (mole) Texas Red-DHPE on the GAPS-coated surfaces after passing through a water/air interface 10 times. FIG. 3C shows fluorescence images of betal GPCR arrays after the binding of 5 nM Bodipy-TMR-CGP12177 in either the absence or presence of 20 μM CGP12177.

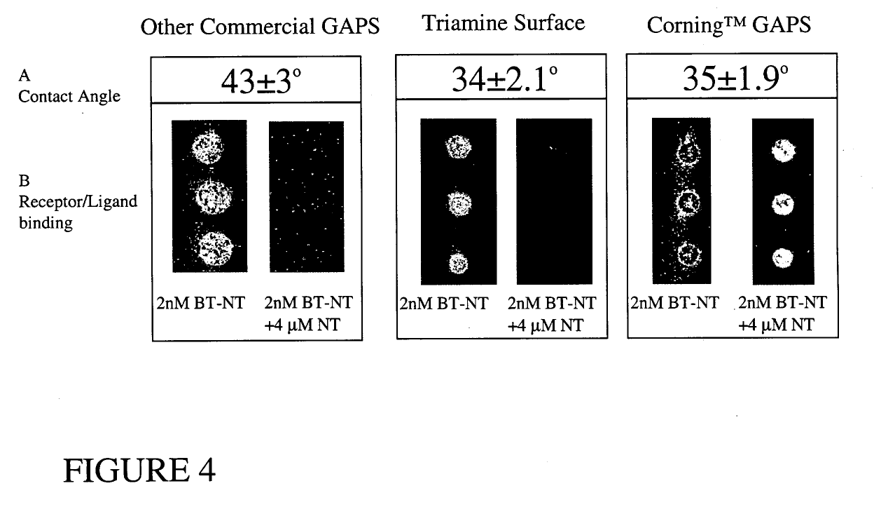

FIG. 4 shows a comparison of three different amine-presenting surfaces. FIG. 4A presents the respective water contact angles. FIG. 4B shows the respective fluorescence images of neurotensin receptor subtype 1 (NTR1) arrays after the binding of 2 nM Bodipy-TMR-neurotensin (BT-NT) in the absence and presence of 4 μM neurotensin (NT).

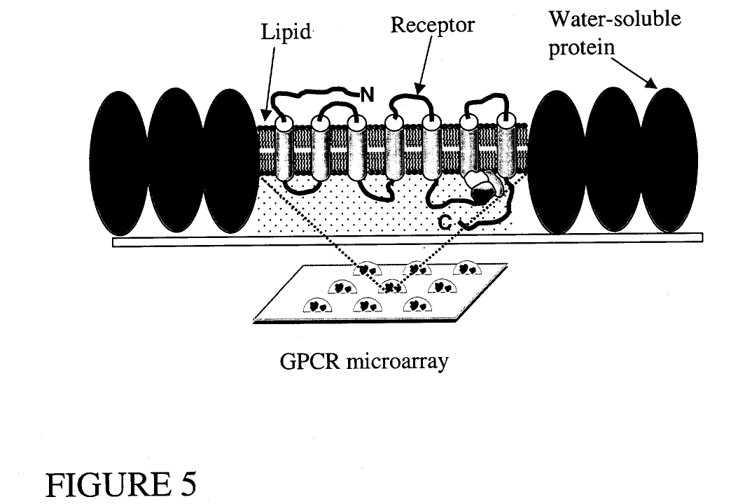

FIG. 5 shows a schematic representation of the use of water-soluble proteins, according to an embodiment such as bovine serum albumin, in the stabilization of biological membrane microarrays.

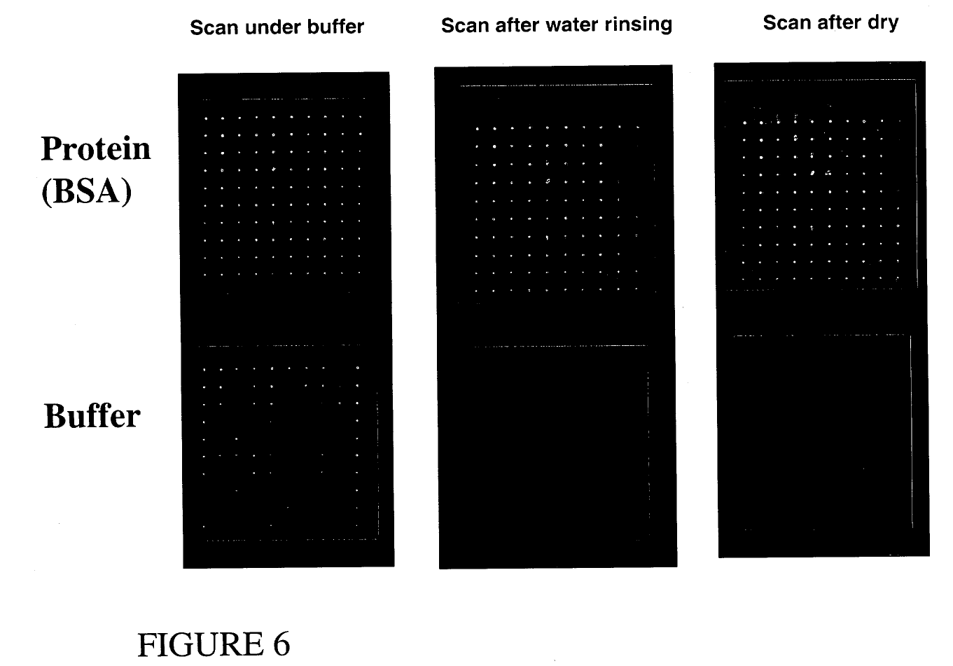

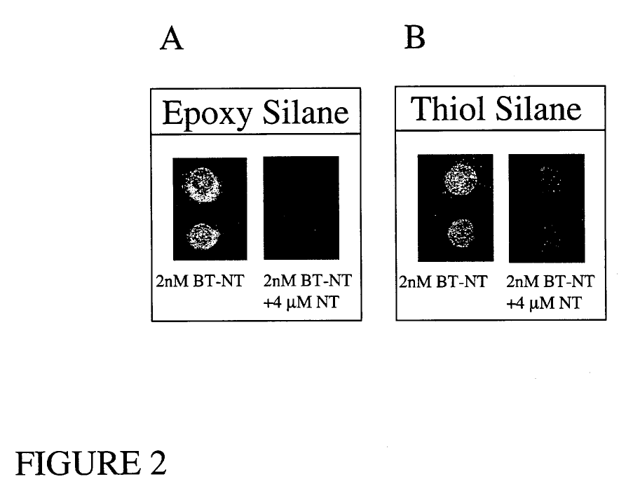

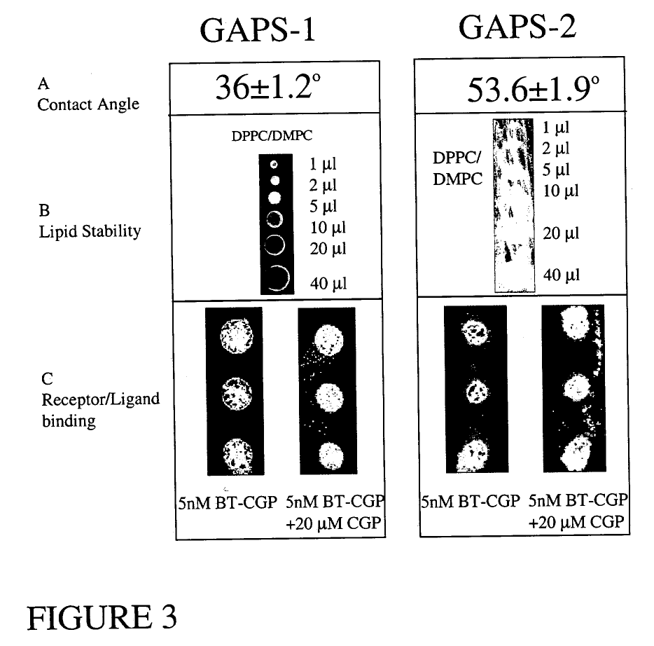

FIG. 6 shows that the presence of BSA significantly increases the stability of lipid arrays against drawing from the water/air interface, solution rinsing and even drying. Two sets of 4×4 arrays of DPPC/DMPC/2% biotin-x-DHPE were printed on Brij-MHA-gold slides. The top set of images was subject to buffer containing 0.1% BSA, and the bottom set was subject to buffer only. After 10 minutes, cy3-streptavidin was introduced and incubated for another 10 minutes. Afterwards the slide was rinsed and imaged under buffer (left), drawn 5 times through a water/air interface and imaged under buffer (middle), and finally dried and imaged again (right). The buffer contained 10 mM phosphate, 1 mM EDTA, 1 mM MgCl₂, pH 7.4.

### DETAILED DESCRIPTION OF THE INVENTION

The invention incorporates a variety of surface chemistries to control the attachment and stability of biological membrane arrays on substrates. Refinement of certain surface chemistry characteristics has enabled the present invention to develop different types of surfaces for improved stability of biological membrane microarrays and to overcome the problems and disadvantages associated with previous arrays. In part, the present invention expands upon research that was described in U.S. Patent Application Publication Nos. 2002/0019015, and 2002/0094544, the contents of both are incorporated herein by reference.

Biological membrane-protein arrays of the present invention are associated with a substrate having either a polar surface or a reactive surface. Several factors significantly affect the spot size, uniformity, stability, functionality and ligand-binding specificity of membrane-protein arrays (particularly for G-protein coupled receptor arrays) and their use in pharmacological ligand binding assays. These factors include the printing conditions, printing ink compositions, surface chemistries, bioassay conditions, and receptor quality. Among these factors, surface chemistry plays a major role in determining the quality and bioassay possibilities of a membrane protein array. The structure and properties of lipid molecules and membrane protein-associated lipids immobilized on a surface strongly depend on the chemical nature of the surface. Hence, several surface chemistries have been designed for immobilizing these species.

Ideally, a surface to be used as a substrate for membrane protein arrays should have the following properties: (1) The size of the microspots should be controllable thus enabling fabrication of an array with a desired density of spots; (2) The printed membrane microspots should be confined in predetermined or designed locations before and after bioassays; (3) The membrane-bound proteins in each microspot should retain their biological functions, and demonstrate specificity and affinities similar to those exhibited in homogenous assays; (4) The printed microspots of biological membranes should be physically stable and resistant to removal from the surface over the course of a bioassay, which may include various preparation and handling treatments, such as incubation with a binding buffer, rinsing with different media, drying, or exposure of the microspots to air during handling; (5) Non specific binding of target molecules should be minimal; (6) Lipids in the printed membrane should retain their lateral fluidity, an intrinsic and physiologically important property of biological membranes. While the importance of preserving long-range fluidity in a supported-membrane microspot for their applications such as compound screening assays is unclear, the ability to preserve long-range fluidity without compromising assay and array performance can be presumed to be advantageous; (7) The biological membranes on the surface should not spread uncontrollably or beyond predetermined tolerances during deposition and immobilization processes. The present invention successfully addresses these issues.

A variety of techniques may be used to produce the array of biological membranes of the invention. For example, arrays of the present invention can be produced using microstamping (U.S. Pat. No. 5,731,152), microcontact printing using PDMS stamps (Hovis 2000), capillary dispensing devices (U.S. Pat. No. 5,807,522), micropipetting devices (U.S. Pat. No. 5,601,980), and any conventional printing technologies such as solid pin printing and quill pin printing that are widely used for the fabrication of DNA and/or protein microarrays.

In the arrays of the present invention, a plurality of biological membrane probe spots is associated with the surface of a solid support. The microspots are associated in a stable fashion with the surface of a substrate. As used herein, the phrase “associated in a stable fashion” refers to microspots maintaining their positions relative to the substrate under binding and/or washing conditions. That is, the microspots remain in location and biological membranes in each microspot retain their biological functions when drawn through an air-water interface. As such, the biological membranes, which make up the spots, can be either covalently or non-covalently associated in a stable fashion with the substrate surface. Examples of covalent binding may include covalent bonds formed between the probe proteins or other non-probe proteins coexisting in the biological membrane spot, and a functional group present on the surface of the substrate, where the functional group may be either naturally occurring or present as a member of an introduced coating material, for instance, an amine reactive group, or a thiol reactive group, or some other electrophilic reactive group. In another example, histidine-tagged mutations of GPCRs or membrane proteins can be used to fabricate microarrays, where the histidine-tag of the membrane proteins can bind to Ni-presenting surfaces through chelating bonds. Examples of non-covalent association may include non-specific physical adsorption, binding based on electrostatic (e.g. ion, ion pair interactions), hydrophobic interactions, hydrogen bonding interactions, surface hydration force and the like, and specific binding based on the specific interaction of an immobilized binding partner and a membrane bound protein. Specific binding-induced immobilization includes, for example, antibody-antigen interactions, generic ligand-receptor binding, lectin-sugar moiety interaction, etc. An example is to use a surface presenting wheat germ agglutinin, which can specifically recognize and bind to glycoslyated residues of the extracellular domains of membrane proteins in the microspots. Another example is to use a surface presenting anti-histidine-tag antibodies or any other anti-“tag” antibody that can bind the histidine tag or any other “tag” of a membrane protein mutant in the biological membrane microsopts. These tagged membrane protein mutants can be produced by state-of-the-art approaches. Also, a surface presenting anti-G-protein antibodies, which selectively bind to specific G-proteins present in the biological membranes of a GPCR, can be used for fabrication of GPCR arrays. The binding between the antibody and specific G-proteins can enrich certain populations of GPCRs, which only couple to the specific G-protein in the membrane preparations.

An array of the present invention may optionally further comprise a coating material on the whole or a portion of the substrate comprising the probe microspots. Preferably the coating material enhances the affinity of the biological membrane microspot for the substrate. In one embodiment, a coating material confers a water contact angle ranging from about 5° to 80°. A preferred coating material confers a contact angle ranging from about 15° to 60°. A most preferred coating material confers a water contact angle ranging from 25° to 45°. Treated substrates having water contacts angles between 25-35 or 45 degrees may be used for high-density arrays; substrates having water contact angles from about 5 degrees up to 30 or 35 degrees may be used for low-density arrays.

Section I. Biological Membrane Arrays on Polymer-Grafted Surfaces

Membrane proteins associated with lipid bilayers generally comprise domains that extend beyond the lipid bilayers (so called extra-membrane domains). After biological membranes are immobilized onto a solid surface, these extra-membrane domains can become misfolded due to physical contact between the protein and the solid surface. To prevent such activity losses of membrane spanning proteins, polymer cushions (surface grafted layers or films of hydrophilic polymers) are often employed as a spacer between the bilayer (and the associated proteins ) and solid support. These polymer cushions not only provide a sufficient hydration layer between the lipids and the surface, they are also deformable. A thicker hydration layer between the surface and lipid bilayer permits the membrane protein to be better integrated in its native environment and retain its functionality. Polymer-cushion surfaces can be used for the immobilization of biological membranes and their applications in biosensor detection. Hydrophilic polymer surfaces, however, have not, as far as we know, been used for fabrication of membrane-protein arrays.

Particular embodiments of the invention are described in terms of hydrophilic polymers. A polymer includes a molecule consisting of more than one monomer unit, such as amino acids, ethyleneimine, etc. Examples of hydrophilic polymers include positively charged polymers, such as poly-lysine, poly-histidine, poly-ethyleneimine, or DEAE-dextran, etc. Alternatively, a hydrophilic polymer may be a lipopolymer, such as poly(ethyloxazoline-co-ethyleneimine-co-pentadecanyloxazoline). In other embodiments, a hydrophilic polymer is a reactive polymer such as maleic anhydride, or a negatively charged polymer such as poly-glutamic acid. A hydrophilic polymer can also be a neutral polymer. According to yet another embodiment, a hydrophilic polymer is a mixture of more than one of the aforementioned type of polymers. Most preferably, amine-presenting polymers are used to coat a substrate for the fabrication of biological membrane microarrays.

In one embodiment, a surface having physically adsorbed layers of a hydrophilic polymer is employed to fabricate a biological membrane microarray using pin-printing techniques. The polymer coating may be applied using state-of-the-art approaches, such as by means of pulsed plasma to deposit layers of polymers onto a solid substrate surface. The polymer coating may result in the formation of a loosely packed polymer layer referred to as a “polymer cushion.”

In another embodiment, a surface presenting amine-reactive functional moieties, such as isothiocyanate, NHS ester, epoxide, and anhydrides, etc., can be modified with a molecule presenting more than one amine groups (e.g., 1,6-hexanediamine) to form an amine-presenting surface. An example of another modification is to react 1,6-hexanediamine with a surface presenting poly(maleic anhydride- co-butylvinyl ether) to form a polymer drafted surface with amine functionality. Similarly, a surface presenting other reactive groups, such as thiol- and carboxylate-reactive groups, can be modified to present at least one amine group.

In an alternative embodiment, the coating material is a derivatized monolayer (or several monolayers), multilayer or polylayer having covalently bonded linker moieties. Most preferably, the monolayer comprises a thioalkyl compound or a silane compound. Preferably, the silane- or thiol-derivatized surface can be further modified with one or more reagents (e.g. cationic polymers such as poly(diallydimethylammonium chloride, or glutaraldehyde) to enable membrane immobilization.

A surface having layers of polymers physically adsorbed or covalently grafted that has a water contact angle between 0° and 30° is preferable for fabrication of low-density microarrays of biological membranes. The term “low-density microarrays,” as used herein refers to a microarray having a number of microspots with diameters larger than 500 μm and separated from each other by more than 500 μm. A surface having layers of polymers physical adsorbed or covantly grafted that has a water contact angle between 25° and 80° (most preferably between 25° and 45°)is preferable for fabrication of biological membrane microarrays.

### EXAMPLE

Neurotensin receptor subtype 1 (human, NTR1) was purchased from Perkin Elmer Life Sciences (Boston, Mass.) and was received as a membrane associated suspension in a buffer. Printing was carried out using a quill-pin printer (Cartesian Technologies Model PS 5000) equipped with software for programmable aspiration and dispensing. After printing the arrays were incubated in a humid chamber at room temperature for one hour, and then used for ligand binding experiments. Two different binding solutions, 2 n-M BT-NT in binding buffer in either the absence or presence of neurotensin, were used to examine the total binding of fluorescently labeled neurotensin (2 nM BT-NT), and the specific inhibition of the binding of BT-NT in the presence of 4 μM unlabeled neurotensin. The binding buffer contained 50 mM Tris-HCl, pH 7.4, 10 mM MgCl₂, 1 mM EDTA, 0.1%BSA. The incubation time for binding was one hour. After incubation, the slides were rinsed, dried, and examined using a Genipix scanner.

Glass slides were coated by incubating the slide with a 3 ml solution containing 1 mg/ml polymer (poly-lysine, 46 kDa, or polyethyleneimine, 50 kDa) in 0.1×PBS buffer for 30 minutes at room temperature, followed by rinsing with water and drying under nitrogen. Two examples are shown in FIGS. 1A and B. The polymer grafted surfaces were hydrophilic as expected. Specifically, the poly-lysine surface had a water contact angle of about 10°, and the polyethyleneimine surface had a water contact angle of 11°. Using a Telechem quill pin CMP5, we fabricated GPCR arrays. The diameter of the GPCR microspots was about 1 mm, indicating that these surfaces are suitable for fabrication of low density arrays of membrane proteins. The binding specificity of BT-NT, indicated by the competitive binding of unlabeled excess neurotensin, was f˜90% for both surfaces.

Section II. Biological Membrane Arrays on Reactive Surfaces

To reiterate, since lateral diffusion of molecules within a cell membrane is a fundamental property of natural biological membranes, covalent immobilization of the entire membrane is not desirable for the fabrication of biological membrane microarrays. In cell membrane preparations of a GPCR, however, many other membrane proteins are associated with the lipid membranes(for example, membrane-bound G proteins, adenylyl- cylases, and receptor tyrosine kinases).

According to the invention, a GPCR-membrane preparation can be immobilized onto a reactive surface through covalent interaction without significant loss of activity of the receptor of interest. The reactive surface has a binding functional moiety or molecule. Preferably, biological membranes are arrayed onto a reactive surface through covalent bonds formed between the probe proteins of interest or other non-probe proteins in the biological membrane spot and a functional group present on the surface of the substrate. The functional group may be an amine reactive group, or a thiol reactive group, or any other reactive group. Alternatively, histidine-tagged mutations of GPCRs or membrane proteins can be used to fabricate microarrays, where the histidine-tag of the membrane proteins can bind to Ni-presenting surfaces through chelating bonds.

The coating material presenting reactive functional groups, according to an embodiment, may be a silane, thiol, disulfide, or a polymer. When the coating material is a silane, the substrate comprises glass. Preferably, the silane presents terminal moieties including, for example, glycidoxy, isocyanato, or thiol groups. Alternatively, silanes presenting functional groups that can be further modified to become reactive can also be used. For example, a silane presenting carboxyl groups is used to coat the surface of a substrate, followed by standard NHS ester activation to form an amine-reactive surface. When the coating material is a thiol, the substrate comprises gold. Preferably, the thiol presents terminal moieties including, for example, thiol, or isocyanato groups. When the thiol presents carboxyl or other functional terminal groups, the thiol-modified surface can be further activated to form reactive surfaces using standard conjugation chemistries. The coating may also involve the use of a polymer that presents reactive groups. For example, a copolymer presenting maleic anhydride, such as poly(maleic anhydride- co-butylvinyl ether), can be used to coat a surface of a substrate.

### EXAMPLE

Neurotensin receptor subtype 1 (human, NTR1) was purchased from Perkin Elmer Life Sciences (Boston, Mass.) and was received as a membrane associated suspension in a buffer. Printing was carried out using a quill-pin printer (Cartesian Technologies Model PS 5000) equipped with software for programmable aspiration and dispensing. After printing the arrays were incubated in a humid chamber at room temperature for one hour, and then used for ligand binding experiments. Two different binding solutions, 2 nM BT-NT in binding buffer in either the absence or presence of neurotensin, were used to examine the total binding of fluorescently labeled neurotensin (2 nM BT-NT), and the specific inhibition of the binding of BT-NT in the presence of 4 μM unlabeled neurotensin. The binding buffer contained 50 mM Tris-HCl, pH 7.4, 10 mM MgCl₂, 1 mM EDTA, 0.1% BSA. The incubation time for binding was one hour. After incubation, the slides were rinsed, dried, and imaged in a fluorescence scanner.

Glass slides were coated by incubating the slide with a 3 ml ethanol solution containing 2% 3-glycidoxypropyl trimethoxysilane (Epoxy silane) or 2% 3-mercaptopropyl trimethoxyosilane for 30 minutes at room temperature, followed by rinsing with ethanol and water, and drying under nitrogen. Two examples are shown in FIG. 2. The two reactive surfaces are moderately hydrophobic, as expected. The expoxy silane surface has a water contact angle of about 20°, and the thiol-silane surface has a water contact angle of about 24°. The Telechem quill pin CMP5 was used to fabricate GPCR arrays. The diameter of the GPCR microspots was ˜250 μm. The binding specificity of BT-NT, indicated by the competitive binding of unlabeled excess neurotensin, was ˜75-90% on two different reactive surfaces, although the total binding signal was relatively low.

Section III. Biological Membrane Microarrays on a Surface with Appropriate Surface Properties

Unlike DNA arrays or other protein arrays, arrays of membrane proteins including GPCRs not only require immobilization of both the targets of interest and the associated lipid molecules, they also require the “right” immobilization. For example, the protein-associated lipid molecules arrayed on a surface should maintain their freedom to move around along the surface. The hydration layer between the surface and lipid bilayer should be sufficiently thick to avoid the misfolding of extra-membrane domains of membrane proteins. Furthermore, for GPCR arrays, the receptor-G protein complexes should be preserved after being arrayed onto the surface. On the other hand, for any robust bioassays using microarrays, the microspots should be mechanically stable and remain confined to the printed location. Performance of membrane protein arrays including GPCR arrays depends on a number of factors, including surface chemistry, membrane content quality, printing quality, and bioassay conditions. Among these factors, surface chemistry plays a major role in determining the quality and bioassay possibilities of a membrane protein array. The structure and properties of lipid molecules and membrane protein-associated lipids immobilized on a surface strongly depends on the chemical nature of the surface.

Among the surfaces considered to date, amine-presenting surfaces have provided the best quality membrane protein binding microarrays. Methods to screen and characterize any given surface for binding of lipids and membrane proteins are important.

In one embodiment, the present invention relates to a method of how to screen and select appropriate surface chemistries for the fabrication of biological membrane microarrays. In another embodiment, the present invention relates to the important properties of a potential surface, which determine the suitability of the surface for biological membrane microarrays.

According to one method, a water contact angle measurement is used to examine the hydrophobicity of an amine-presenting surface. The hydrophobicity has a dramatic effect on immobilization kinetics, degree of spot-spreading and mechanical stability of biological membranes, and the non-specific binding of labeled probe proteins or ligands during the performance of bioassays. Preferably, a solid substrate is coated with a material that provides appropriate hydrophobicity. In one embodiment, a coating material confers a water contact angle ranging from about 5° to about 80°. A preferred coating material confers a contact angle ranging from about 15° to about 60°. A more preferred coating material confers a water contact angle ranging from about 25° or 35° to about 40° or 45°, wherein the surface is suitable for fabricating membrane protein microarrays of medium to high densities. In other embodiments, a coating material that confers a water contact angle between 0° or 5° and 25° is preferred for low-density arrays. “Low-density,” as used herein in general, refers to fewer than about 100-110 microspots per cm². In a particular embodiment, one spot of membrane protein per well is preferably created in any given microplate format, such as 96-, 384-, or 1536-wells, etc.

In another method, a lipid stability measurement is used to examine the morphology of a series of lipid spots on a surface before and after the surface is withdrawn through a water/air interface a certain number of times. The lipid spot stability provides reliable information about the mechanical stability of biological membrane microarrays on the surface, and thereby the suitability of the surface for biological membrane microarrays. Preferably, a surface is coated with a material that enhances the stability of lipid spots against withdrawl through an water/air interface and washing and drying.

In a further method, ligand-binding specificity is examined to evaluate the immobilization of the biological membrane arrays on a substrate. The quality and functionality of membrane-protein microarrays can be controlled with a proper surface chemistry. A surface coated with a material, which gives rise to minimal non-specific binding of labeled targets to the background, and high specific binding to the probe receptors in the microarrays is preferred.

Most preferably, any combination of the above three methods may be used to screen and select an appropriate surface chemistry for biological membrane microarrays. FIGS. 3 and 4 presents examples that show the correlation among contact angle, lipid stability and ligand binding to membrane receptors in the arrays for amine presenting surfaces. Table 1 summarizes the results for other examples.

TABLE 1

| | Ex. 1 | | Ex. 2 | | Ex. 3 | | Ex. 4 | |
|---|---|---|---|---|---|---|---|---|
| | a | b | a | b | a | b | a | b |
| Contact angles | 43.8 ± 1.5 | 42.1 ± 1.5 | 54.8 ± 1.3 | 57.5 ± 1.7 | 67.3 ± 1.7 | 68.3 ± 2.6 | 46.5 ± 4.2 | 49 ± 1.8 |
| Lipid Stability | | | | | | | | |
| Eye Inspection | +^{a} | + | − | − | − | − | + | + |
| Imaging | −^{b} | − | − | − | + | + | − | − |
| Ligand Binding | | | | | | | | |
| Signal | 7486 ± 820 | 7996 ± 1786 | 8086 ± 893 | 6828 ± 348 | 5822 ± 993 | 5721 ± 838 | 9187 ± 2192 | 7881 ± 1337 |
| Specificity | 46% | 42% | 49% | 50% | 31% | 38% | 43% | 33% |
| S/N ratio* | 5.3 | 4.6 | 5.0 | 4.4 | 3.3 | 3.3 | 5.4 | 7.4 |
| Background | 605 | 345 | 1422 | 1430 | 2012 | 2035 | 392 | 365 |
| True S/N | 12 | 23 | 5 | 5 | 3 | 3 | 23 | 22 |

| | Ex. 5 | | Ex. 6 | | Ex. 7 | | Ex. 8 | |
|---|---|---|---|---|---|---|---|---|
| | a | b | a | b | a | b | a | b |
| Contact angles | 41.5 ± 1.9 | 41.5 ± 1.8 | 40.4 ± 1.1 | 39.2 ± 1.2 | 40.8 ± 0.6 | 40.0 ± 1.1 | 53.6 ± 1.9 | 53.3 ± 2.8 |
| Lipid Stability | | | | | | | | |
| Eye Inspection | ++ | ++ | ++ | ++ | ++ | ++ | − | − |
| Imaging | ++ | ++ | ++ | ++ | ++ | ++ | − | − |
| Ligand Binding | | | | | | | | |
| Signal | 9241 ± 1060 | 9658 ± 807 | 6003 ± 539 | 7932 ± 900 | 11029 ± 1639 | 10513 ± 2334 | 4772 ± 1868 | 5461 ± 688 |
| Specificity | 30% | 34% | 37% | 32% | 46% | 42% | 18% | 44% |
| S/N ratio* | 9.6 | 9.6 | 7.4 | 11.1 | 6.6 | 12.5 | 3.6 | 3.4 |
| Background | 208 | 212 | 228 | 213 | 215 | 213 | 1450 | 1531 |
| True S/N | 44 | 46 | 26 | 37 | 51 | 49 | 3 | 3 |

S/N Ratio = (I_{positive control} + I_{background})/I_{background} (First column)
Specificity = (I_{positive control} − I_{inhibition)}/I_{positive control}
Signal - mean fluorescence intensity of the first colunm (background substraction)
True S/N = (I_{positive conrrol}/I_{background}) (using true background)

According to one embodiment, the coating material is a derivatized monolayer or multilayer having covalently bonded linker moieties. The monolayer coating, for example, comprising long chain hydrocarbon moieties, may have for example, but not limited to, thiol (e.g., thioalkyl), disulfide or silane groups that produce a chemical or physicochemical bonding to the substrate. The attachment of the monolayer to the substrate may also be achieved by non-covalent interactions or by covalent reactions.

After attachment to the substrate the layer has at least one reactive functional group. Examples of reactive functional groups on the coating include, but are not limited to, carboxyl, isocyanate, halogen, amine or hydroxyl groups. In one embodiment, these reactive functional groups on the coating may be activated by standard chemical techniques to corresponding activated functional groups on the coating (for example, conversion of carboxyl groups to anhydrides or acid halides, etc.). The activated functional groups of the coating on the substrate may be, but not limited to, anhydrides, N-hydroxysuccinimide esters or other common activated esters or acid halides, for covalent coupling to terminal amino groups of the linker compound. In another embodiment, the activated functional groups on the coating may be, but not limited to, anhydride derivatives for coupling with a terminal hydroxyl group of the linker compound; hydrazine derivatives for coupling onto oxidized sugar residues of the linker compound; or maleimide derivatives for covalent attachment to thiol groups of the linker compound. To produce a derivatized, coating, at least one terminal carboxyl group on the coating layer is first activated to an anhydride group and then reacted with a linker compound.

Alternatively, the reactive functional groups on the coating may be reacted with a linker compound having activated functional groups, for example, but not limited to, N-hydroxysuccinimide esters, acid halides, anhydrides, and isocyonates for covalent coupling to reactive amino groups on the monolayer coating.

The linker compound has one terminal functional group, a spacer region and a membrane adhering region. The terminal functional groups for reacting with the activated functional groups on the activated monolayer coating are for example, but not limited to, halogen, amino, hydroxyl, or thiol groups. Preferably, the terminal functional group is selected from the group consisting of a carboxylic acid, halogen, amine, thiol, alkene, acrylate, anhydride, ester, acid halide, isocyanate, hydrazine, maleimide and hydroxyl group.

The spacer region may consist of, but not limited to, oligo/poly ethers, oligo/poly peptides, oligo/poly amides, oligo/poly amines, oligo/poly esters, oligo/poly saccharides, polyols, multiple charged species or any other combinations thereof. Examples include, but are not limited to, oligomers of ethylene glycols, peptides, glycerol, ethanolamine, serine, inositol, etc., and are such that membranes freely adhere to the membrane adhering region of the linker moiety. The spacer region may be hydrophilic in nature. In one preferred embodiment, the spacer has n oxyethylene groups, where n is between 2 and 25. In the most preferred embodiment, the spacer has ten oxyethylene groups. In a preferred embodiment the membrane adhering region or “hydrophobic tail” of the linker compound is hydrophobic or amphiphilic with straight or branched chain alkyl, alkynyl, alkenyl, aryl, araalkyl, heteroalkyl, heteroalkynyl, heteroalkenyl, heteroaryl, or heteroalkynyl. In a preferred embodiment, the membrane adhering region comprises a C₁₀ to C₂₅ straight or branched chain alkyl or heteroalkyl hydrophobic tail. In the most preferred embodiment, the hydrophobic tail comprises a C₁₀ to C₂₀ straight or branched chain alkyl fragment, such as C₁₈.

In another embodiment, the linker compound has a terminal functional group on one end, a spacer, a linker/membrane adhering region and a hydrophilic group on another end. The hydrophilic group at one end of the linker compound may be a single group or a straight or branched chain of multiple hydrophilic groups. For example, but not limited to, a single hydroxyl group or a chain of multiple ethylene glycol units.

In a further embodiment, the “derivatized monolayer” is a self-assembled monolayer (SAM) of an alkanethiol modified with a silane. Alkanethiols preferably include, for example, 11-mercaptoundecanol (MUD), 11-mercaptoundecanoic acid (MUA), 11-mercaptoundecylamine (MUAM), 16-mercaptohexadecanol, and 16-mercaptohexadecanoic acid. Silanes preferably include silanes with different terminal functional groups as specified earlier, including 3-amino-propyl-trimethoxysilane (APTES), 3-mercapto-propyl-trimethoxysilane, and 3-isocyanatopropyltriethoxysilane. In this embodiment, the substrate preferably comprises a gold surface. The use of a substrate comprising a gold surface results in enhanced signal to background ratios compared to arrays printed on glass substrates. Additionally, gold is a preferred substrate for label-free detection techniques including surface plasma resonance (SPR), matrix assisted laser desorption ionization mass spectrometry (MALDI-MS) and electrochemical methods.

### EXAMPLES

Method 1: Contact Angle Measurements.

CMT GAPS slides from different lots were used as received. Amine presenting slides from competitors, Xenoslide A slide (aminosilane surface) from Xenopore Inc., Starsoft slides (aminosilane surface) from Sigma, DNA ready slides from Clontech, Aminosilane slides from Telchem, were used as received. A triamine surface was formed by silanizing a clean glass slide with 3-(2-(2-aminoethylamino)-ethylamino)propyl trimethyloxysilane using standard silanization procedures. An amine presenting gold surface was fabricated by formation of a SAM of 11-mercaptoundecylyamine (MUAM) for one hour (MUAM-Au1h) or 24 hours (MUAM-Au1D).

A water contact angle measurement was carried out at 4 different locations of each of the two slides examined (see FIG. 4).

Method 2: Lipid Spot Stability.

A surface on which lipids are associated stably is preferred for fabricating membrane arrays. To evaluate lipid stability on surfaces, several lipid spots of different sizes were formed by means of depositing solutions containing vesicles of synthetic lipids doped with fluorescently labeled lipids. A lipid solution containing DLPC and 2% (mole) Texas Red-DPHE was used after sonication to form lipid spots of different sizes on these surfaces by transferring different amounts of solution, ranging from 0.5 to 40 μl. After deposition, the surface with the lipid spots was incubated for one hour at room temperature at ˜95% relative humidity. After incubation, excess solution was removed, and the surface was rinsed with a flow of water for a several seconds, followed by a quick withdrawl through a water/air interface several times (10), and dried. The stability of the lipid spots was examined in two ways. First, using the naked eye, water condensation around the spots during the extraction was examined. Lipid spots are stable if water drops condense around the spots. Second, the lipid spots were imaged using a fluorescence scanner to examine the fluorescent intensity of the lipid spots. The lipid spots are stable if the fluorescence intensities of the spots are strong and uniform.

Method 3: Receptor-ligand binding.

Human neurotensin receptor subtype 1 (NTR1) and β-adrenoreceptor subtype 1 (β1) were purchased from Perkin Elmer Life Sciences (Boston, Mass.) and received as a membrane associated suspension in a buffer. Printing was carried out using a quill-pin printer (Cartesian Technologies Model PS 5000) equipped with software for programmable aspiration and dispensing. After printing, the arrays were incubated in a humid chamber at room temperature for one hour, and then used for ligand binding experiments.

FIGS. 3A-C compare two different GAPS slides in terms of hydrophobicity, lipid spot stability and ligand binding specificity. FIGS. 4A-C compare three different amine-presenting surfaces in terms of hydrophobicity and ligand binding specificity. The results reveal that a correlation exists between hydrophobicity, lipid spot stability and ligand binding specificity as well as array performance, even when the surface is coated with the same material.

Section IV. Biological Membrane Microarrays Stabilized by Water-Soluble Proteins

Another aspect of the present invention relates to a method to stabilize lipid and membrane protein-associated lipid microarrays using reagents that include generic, water-soluble proteins. Generic proteins may include any water-soluble protein that will not interfere with the binding domains of target membrane proteins or other functional molecules arrayed onto surfaces. Examples of such generic proteins may include bovine serum albumin (BSA), which can bind non-specifically to substrates such as bare glass, mica, gold, self-assembly monolayers of silanes and alkylthiols, and polymer-grafted surfaces.

In some cases the generic proteins may form highly packed monolayers on the surface. These surface-bound proteins remain hydrated during the drying of the surfaces. Given that for certain kinds of substrates exposure to air destroys the supported lipid membranes, this feature of the proteins is a great benefit. Hence, according to the invention, these proteins are employed to form dense layers surrounding lipid and/or lipid/membrane protein spots. These protein layers significantly reduce the risk of biological membrane microarrays being exposed to air, and minimize damage to biological membranes caused by hydrodynamic forces when the membrane microarrays are withdrawn through water/air interfaces during rinsing and washing steps. Moreover, the proteins may serve to stabilize and anchor hydrophobic lipid bilayers by surrounding the edges of biological membrane microspots, which normally are susceptible to damage. FIG. 5 is a schematic representation that illustrates the concept.

### EXAMPLE

Lipid microarrays were generated using either a contact printing technique (e.g., quill pin) or manual spotting on a slide surface, and incubated in a humid chamber at room temperature. Afterwards, the lipid microarrays were covered by buffer with or without generic proteins (between ˜0.01% and 1%), and incubated for ten minutes. The lipid arrays were then rinsed with buffer to remove unbound proteins.

To evaluate the stability of the lipid arrays, the printed slides were drawn through a water/air interface several times, and then imaged in a fluorescence microscope or a scanner. To evaluate the functionality of lipid/protein arrays, fluorescent dye-labeled probe molecules were introduced into the solution, followed by rinsing to remove unbound molecules, drying, and imaging.

Arrays of lipids containing either 1% (mol) Texas Red-DHPE or 2% biotin-x-DHPE were printed onto bare glass slides, Corning GAPS slides and Brij-MHA-gold surfaces. On each slide there were two 4×4 arrays. One array was covered with buffer containing 0.1% BSA, while the second array was covered with buffer in the absence of BSA. After 10 minutes these slides were drawn through the water/air interface 5 times. During this drawing process it was found that in almost every case the presence of BSA helped in the formation of water droplets around lipid spots. After drawing the slides through the interface, the lipid arrays were covered again by buffer and further examined in a fluorescence microscope or scanner. The results presented in FIG. 6 show that the presence of BSA protects the lipid arrays from being removed from the surfaces. These observations suggest that the adsorbed BSA molecules increase the mechanical stability of the printed lipid arrays.

The specific binding of streptavidin to biotin molecules was used as a model system to examine the effect of BSA adsorption on the accessibility and/or functionality of lipid arrays. Seven 5×5 arrays of DPPC/DMPC/2% biotin-x-DHPE were printed onto a single Corning GAPS slide (data not shown). After incubation 3 sets of lipid arrays were pre-incubated with TR-BSA before the addition of Cy3-streptavidin. A second set of 3 arrays was incubated with TR-BSA together with cy3-streptavidin, while one array was covered with a solution of cy3-streptavidin. After 30 minutes the slides were rinsed, dried and imaged. The cy5 channel signal was used to monitor the adsorption of TR-BSA, and the cy3 channel signal was used to monitor the binding of cy3-streptavidin. The results show that, although there is some minimal amount of non-specific binding of BSA to lipid spots, the presence of BSA (concentrations of 0.01%-1%) does not significantly decrease the binding specificity. Simultaneously the presence of BSA reduces the background level in most cases.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

We claim:

1. A method for fabricating a biological membrane array on a surface, the method comprises: providing a support substrate; creating either a polar surface or reactive surface on said support; providing a solution of biological membranes; and depositing an array of biological-membrane microspots on said support, wherein said microspots are associated in a stable fashion with the surface of said support.

2. The method according to claim 1, wherein said microspots remain in defined locations and retain their biological functions in either a liquid or air environment, or both.

3. The method according to claim 1, wherein said deposition is by printing under either ambient or controlled humidity conditions.

4. The method according to claim 1, further comprises applying a reagent to stabilize said biological membranes on said support.

5. The method according to claim 4, wherein said reagent may include a hydrophilic or charged polymer.

6. The method according to claim 5, wherein said polymer is carboxymethyldextran.

7. The method according to claim 4, wherein said reagent may include a soluble protein.

8. The method according to claim 4, wherein said reagent may include water-soluble proteins that will not interfere with binding domains of target membrane proteins or other functional molecules in said biological membrane on said support.

9. The method according to claim 8, wherein said protein is bovine serum albumin (BSA).

10. The method according to claim 8, wherein said proteins on said substrate pack together closely to form at least a layer around said biological membrane microspots, thereby stabilizing said biological membrane arrays.

11. The method according to claim 1, wherein said biological membrane microspots comprise membrane proteins, including either a G-protein coupled receptor (GPCR), a G-protein, an ion channel, a receptor serine/threonine kinase, a receptor guanylate cyclase or a receptor tyrosine kinase.

12. The method according to claim 11, wherein when said biological membrane microspot comprises a GPCR, the GPCR may be oriented depending on the use of said array.

13. The method according to claim 1, wherein said substrate can comprise a glass, silicon, metal, or polymeric material.

14. The method according to claim 1, wherein said substrate is configured as a chip, a slide or a microplate.

15. The method according to claim 1, wherein said substrate is coated with a material that confers a water contact angle ranging from about 5° to about 80°.

16. The method according to claim 15, wherein said substrate is coated with a material that confers a water contact angle ranging from about 15° to about 60°.

17. The method according to claim 15, wherein said substrate is coated with a material that confers a water contact angle ranging from about 25° to about 45°.

18. The method according to claim 1, wherein said substrate is coated with a material that confers a water contact angle between 0° and about 25°.

19. The method according to claim 18, wherein said coated substrate is for a low-density array of less than about 100-110 microspots per cm².

20. The method according to claim 1, wherein said substrate is coated with a material that either: (1) enhances the stability of lipid spots during withdrawl through a water/air interface and washing and drying protocols; or (2) gives rise to minimal non-specific binding of a labeled target to said surface, and high specific binding to a probe receptor in a microspot of said membrane array; or (3) both.

21. The method according to claim 1, wherein said substrate is coated with a material selected from a silane, a thiol, a biological or synthetic polymer.

22. The method according to claim 21, wherein when said coating material is a silane, the substrate comprises a glass surface.

23. The method according to claim 22, wherein said coating material is a silane presenting amine functional groups.

24. The method according to claim 23, wherein said coating material is γ-aminopropylsilane.

25. The method according to claim 21, wherein when said coating material is a thiol, the substrate comprises a gold-coated surface.

26. The method according to claim 25, wherein when said coating material is a thiol, the thiol comprises hydrophobic and hydrophilic moieties.

27. The method according to claim 21, wherein when said coating material is a thioalkyl compound that presents at least one polar moiety.

28. The method according to claim 21, wherein when said coating material is a polymer, the polymer presents amine functional moieties.

29. The method according to claim 28, wherein when said coating material is a polymer, said amine functional moieties are either poly-ethyleneimine or poly-lysine.

30. The method according to claim 21, further comprises modifying a given surface with a coating material comprising either a monolayer or multilayer having covalently bonded linker moieties.

31. The method according to claim 1, wherein said reactive surface has an amine-reactive group, a thiol-reactive group, or a binding functional moiety or molecule.

32. The method according to claim 31, wherein said amine-reactive group is an glycidoxy group, an isocyanato group, an anhydride group, or a NHS ester group.

33. The method according to claim 31, wherein said reactive surface may be created by applying a coating having a binding functional moiety that specifically binds to biomolecules in biological membranes.

34. The method according to claim 33, wherein said binding functional moiety or molecule is a wheat germ agglutinin, an anti-G protein antibody, or an anti-his antibody.

35. The method according to claim 1, wherein one microspot of membrane protein is created in any given microplate format.

36. A method for fabricating a stable biological membrane array on a surface, the method comprising: providing a substrate; creating either a polar surface or reactive surface by coating said substrate with a material that either: (1) enhances the stability of lipid spots during withdrawing through a water/air interface and washing and drying protocols; or (2) gives rise to minimal non-specific binding of a labeled target to said surface, and high specific binding to a probe receptor in said membrane array, or (3) both; depositing an array of biological-membrane microspots on said substrate.

37. The method according to claim 36, wherein said microspots remain in defined locations and retain their biological functions in either a liquid or air environment, or both.

38. The method according to claim 36, wherein said method further comprises applying a reagent that includes a protein to stabilize said biological membranes on said surface.

39. The method according to claim 38, wherein said reagent may include a hydrophilic or charged polymer.

40. The method according to claim 38, wherein said reagent may include water-soluble proteins that will not interfere with binding domains of target membrane proteins or other functional molecules in said biological membrane on said support.

41. The method according to claim 40, wherein said protein is bovine serum albumin (BSA).

42. The method according to claim 38, wherein said proteins on said substrate pack together closely to form at least a layer around said biological membrane microspots, thereby stabilizing said biological-membrane arrays.

43. The method according to claim 36, wherein said biological-membrane microspots comprise membrane proteins, including either a G-protein coupled receptor (GPCR), a G-protein, an ion channel, a receptor serine/threonine kinase, a receptor guanylate cyclase or a receptor tyrosine kinase.

44. The method according to claim 43, wherein when said biological-membrane microspot comprises a GPCR, the GPCR may be oriented depending on the use of said array.

45. The method according to claim 36, wherein said substrate can comprise a glass, silicon, metal, or polymeric material.

46. The method according to claim 36, wherein said substrate is configured as a chip, a slide or a microplate.

47. The method according to claim 36, wherein said substrate is coated with a material that confers a water contact angle ranging from about 5° to about 80°.

48. The method according to claim 47, wherein said substrate is coated with a material that confers a water contact angle ranging from about 15° to about 60°.

49. The method according to claim 47, wherein said substrate is coated with a material that confers a water contact angle ranging from about 25° to about 45°.

50. The method according to claim 36, wherein said substrate is coated with a material that confers a water contact angle between 0° and about 25°.

51. The method according to claim 50, wherein said coated substrate is for a low-density array of less than about 110 microspots per cm².

52. The method according to claim 36, wherein an array of microspots of membrane proteins is created in any given microplate format.

53. An article for a biological membrane array comprising: a support substrate; an array of biological-membrane microspots deposited on said support; and either a polar surface or reactive surface on said support, wherein said microspots are associated in a stable fashion with said surface of said support.

54. The article according to claim 53, wherein said microspots remain in defined locations and retain their biological functions in both a liquid and air environment.

55. The article according to claim 53, wherein said biological membrane includes membrane proteins.

56. The article according to claim 53, further comprising a reagent including a protein to stabilize said biological membranes on said support.

57. The article according to claim 56, wherein said reagent may include a hydrophilic or charged polymer.

58. The article according to claim 57, wherein said polymer is carboxymethyldextran.

59. The article according to claim 56, wherein said reagent may include water-soluble proteins that will not interfere with binding domains of target membrane proteins or other functional molecules in said biological membrane on said support.

60. The article according to claim 59, wherein said proteins is bovine serum albumin (BSA).

61. The article according to claim 56, wherein said proteins on said substrate pack together closely to form at least a layer around said biological-membrane microspots, thereby stabilizing said biological-membrane arrays.

62. The article according to claim 53, wherein said biological-membrane microspots comprise membrane proteins, including either a G-protein coupled receptor (GPCR), a G-protein, an ion channel, a receptor serine/threonine kinase, a receptor guanylate cyclase or a receptor tyrosine kinase.

63. The article according to claim 62, wherein when said biological membrane microspot comprises a GPCR, the GPCR may be oriented depending on the use of said array.

64. The article according to claim 53, wherein said substrate can comprise a glass, silicon, metal, or polymeric material.

65. The article according to claim 53, wherein said substrate is configured as a chip, a slide or a microplate.

66. The article according to claim 53, wherein said substrate is coated with a material that confers a water contact angle ranging from about 5° to about 80°.

67. The article according to claim 66, wherein said substrate is coated with a material that confers a water contact angle ranging from about 15° to about 60°.

68. The article according to claim 66, wherein said substrate is coated with a material that confers a water contact angle ranging from about 25° to about 45°.

69. The article according to claim 53, wherein said substrate is coated with a material that confers a water contact angle between 0° and about 25°.

70. The article according to claim 69, wherein said coated substrate is for a low-density array of less than about 110 microspots per cm².

71. The article according to claim 53, wherein said substrate is coated with a material that either: (1) enhances the stability of lipid spots during withdrawl through a water/air interface and washing and drying protocols; or (2) gives rise to minimal non-specific binding of a labeled target to a surface, and high specific binding to a probe receptor in said membrane array; or (3) both.

72. The article according to claim 53, wherein said substrate is coated with a material selected from a silane, a thiol, a biological or synthetic polymer.

73. The article according to claim 72, wherein when said coating material is a silane, the substrate comprises a glass surface.

74. The article according to claim 72, wherein when said coating material is a silane presenting amine functional groups.

75. The article according to claim 74, wherein said coating material is γ-aminopropylsilane.

76. The article according to claim 72, wherein when said coating material is a thiol, the substrate comprises a gold-coated surface.

77. The article according to claim 72, wherein when said coating material is a thiol, the thiol comprises hydrophobic and hydrophilic moieties.

78. The article according to claim 72, wherein when said coating material is a thioalkyl compound that presents at least a polar moiety.

79. The article according to claim 72, wherein when said coating material is a polymer, polymer presents amine functional moieties.

80. The article according to claim 79, wherein when said coating material is a polymer, said amine functional moieties are either poly-ethyleneimine or poly-lysine.

81. The article according to claim 53, wherein said reactive surface comprises an amine-reactive group, a thiol reactive group, or other electrophile group.

82. The article according to claim 81, wherein said amine-reactive group is an glycidoxy group, an isocyanato group, an anhydride group, or a NHS ester group.

83. The article according to claim 53, wherein said reactive surface may be created by applying a coating having a binding functional moiety or molecule that specifically binds to biomolecules in biological membranes.

84. The article according to claim 83, wherein said binding functional moiety or molecule is a wheat germ agglutinin, an anti-G protein antibody, or an anti-his antibody.
